(19) United States
(12) Patent Application Publication
(10) Pub. No.: US 2013/0066287 A1
(43) Pub. Date: Mar. 14, 2013

(54) FEMALE URINATION DEVICE

(71) Applicant: Vincent VALENTI, Brooklyn, NY (US)

(72) Inventor: Vincent VALENTI, Brooklyn, NY (US)

(21) Appl. No.: 13/229,578

(22) Filed: Sep. 9, 2011

(51) Int. Cl. A61F 5/455

(52) U.S. Cl. 604/347

(57) ABSTRACT

The female urination device assists a female in urinating from a standing position and directs the urine a suitable distance from the female. The device comprises a first end having an opening for discharging urine and a second end having an opening for receiving the urine. The opening in the second end is larger than the opening in the first end. The device further comprises a splash guard extending downward from an upper portion of the second end. The splash guard covers a portion of the opening in the second end to provide a sealed perimeter at the second end to contain the urine within the device and prevent leakage of the urine.

### TECHNICAL FIELD

The claimed invention relates to female urination device, and more particularly, to female urination devices which allow a female to urinate from a standing position.

### BACKGROUND OF THE INVENTION

Public restrooms for women are very often unclean and unsanitary. The toilet seats in public restrooms are often dirty or otherwise in an unsanitary condition. Because of this, women often squat above the toilet seat when using the public restroom to avoid touching the toilet seat. This can be difficult and unpleasant way for a woman to urinate, especially for a young girl. Similarly, women may have no choice but to squat while holding her clothing out of the way in order to urinate when no restroom is available while traveling, hiking or camping.

In prior art, various types of female urination devices have been proposed. However, many of these devices are complicated and cumbersome to use. Also, none of these devices provide a splash guard to protect the user from accidental leaking or spillage during use and a cushioned reared opening to alleviate any discomfort or irritation to the user's body during use. Further, none of these devices provide a rough outer surface, e.g., gripping surface, to prevent the device from slipping from the user's hand during use. This is a particular problem because many of these devices are constructed bio-degradable paper that can be flushed in a toilet, see, e.g., U.S. Pat. No. 5,480,703.

Additionally U.S. Pat. No. 5, 966,748 requires the user to wrap a flat sheet of material to form a funnel shaped device and the sanitary pad is susceptible to contamination during use because of its proximity to the anal area.

Further U.S. Pat. No. 4,681,573 requires two hands to prepare the collapsible device for use. One hand to hold the device while using the other hand to push the top portion of the device upward and the bottom portion of the device downward, or alternatively, one hand to push the top portion of the device upward and the other hand to push the bottom portion of the device downward. Also, U.S. Pat. No. 2,878,486 requires two hands to open the device for use from its unfolded position. The device is held in an opened hand with thumb and fingers and the handle portions are pulled in an upward direction with the fingers of the other hand until opened. It is preferable that only one hand is required to use and operate device.

### OBJECT AND SUMMARY OF THE INVENTION

Therefore, it is an object of the claimed invention to solve the aforementioned problems with the conventional female urination device.

Another object of the claimed invention is to provide a female urination device which is simple, easy to use and convenient to carry.

In accordance with an exemplary embodiment of the claimed invention, the female urination device for assisting a female in urinating from a standing position and for directing the urine a suitable distance from the female, comprises a first end having an opening for discharging urine, a second end having an opening for receiving the urine and being larger than the opening in the first end, and a splash guard extending downward from an upper portion of the second end. The splash guard covers a portion of the opening in the second end to provide a sealed perimeter at the second end to contain the urine within the device and prevent leakage of the urine.

In accordance with an exemplary embodiment of the claimed invention, the female urination device as aforesaid further comprises a rolling part to provide a smooth second end to eliminate sharp edges.

In accordance with an exemplary embodiment of the claimed invention, the female urination device as aforesaid further comprises a gripping portion on the exterior surface of the device.

In accordance with an exemplary embodiment of the claimed invention as aforesaid further comprises a sanitary wipe removably attached to an exterior surface of the device.

In accordance with an exemplary embodiment of the claimed invention, he splash guard is located at an upper portion of the second end and a rolling part is located at a lower portion of the second end to eliminate sharp edges.

In accordance with an exemplary embodiment of the claimed invention as aforesaid, the lower portion of the second end is concave in shape to provide a tight contoured fit.

In accordance with an exemplary embodiment of the claimed invention as aforesaid further comprises a second splash guard extending upwardly from the second end to contain the urine within the device and prevent leakage of the urine.

In accordance with an exemplary embodiment of the claimed invention as aforesaid, the female urination device is made from a biodegradable and water-resistant material.

In accordance with an exemplary embodiment of the claimed invention as aforesaid, the interior surfaces of the device are coated with water-resistant coating, epoxy or synthetic resin.

In accordance with an exemplary embodiment of the claimed invention as aforesaid, the device is funnel shaped in an open useable configuration.

In accordance with an exemplary embodiment of the claimed invention, a disposable device for assisting a female in urinating from a standing position and for directing the urine a suitable distance from the female comprises a first end having an opening for discharging urine, a second end having an opening for receiving the urine and being larger than the opening in the first end, and a rolling part at the second end to provide a smooth second end to eliminate sharp edges. Preferably, the device further comprises a splash guard extending downward from an upper portion of the second end and covering a portion of the opening in the second end to provide a sealed perimeter at the second end to contain the urine with the device and prevent leakage of the urine.

Various other objects, advantages and features of the claimed invention will become readily apparent from the ensuing detailed description. Novel features will be emphatically pointed out in the appended claims.

### BRIEF DESCRIPTION OF THE DRAWINGS

The claimed invention is further explained in the description which follows with reference to the drawings, illustrations illustrating, by way of non-limiting examples, various embodiments of the invention, with like reference numerals representing similar parts throughout the several views, and wherein:

FIG. 1 shows a side view of the device with a splash guard and a smooth second end in a closed or flat configuration in accordance exemplary embodiment of the claimed invention;

FIG. 2 shows a perspective view of the device with a dual splash guard in an open or useable configuration with in accordance with an exemplary embodiment of the claimed invention; and

FIG. 3 shows a perspective view of the device with a splash guard deployed and a smooth second end in an open or useable configuration in accordance with an exemplary embodiment of the claimed invention.

### DETAILED DESCRIPTION OF THE EMBODIMENTS

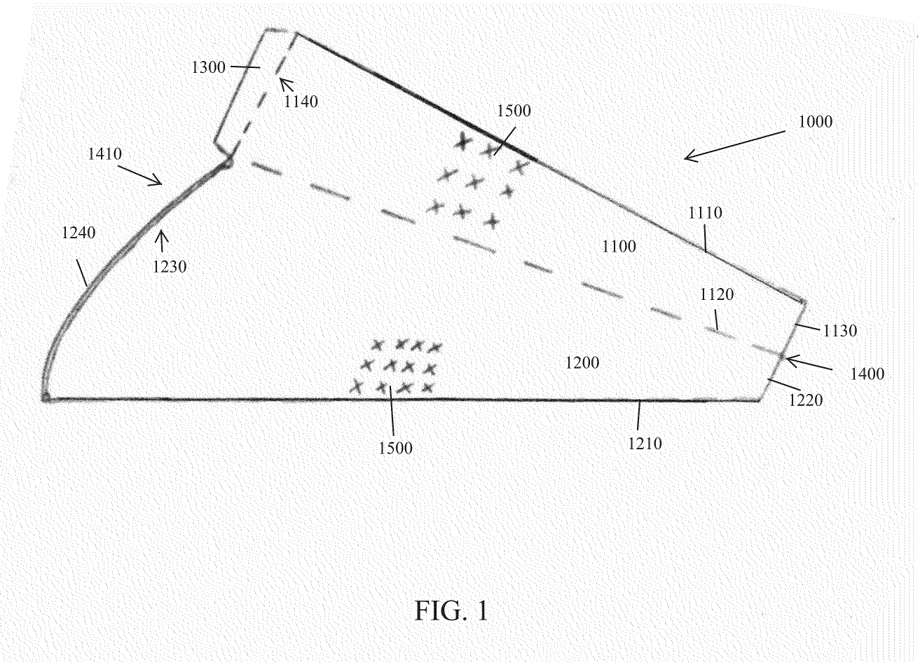

As illustrated in FIGS. 1-3, the female urination device 1000 is designed to assist a female to urinate in a standing position, thereby enabling the female to comfortably and safely urinate while minimizing hygiene risks under any conditions, such as unsanitary restrooms and no available public facilities.

In accordance with an exemplary embodiment of the claimed invention, the device 1000 comprises two opposing upper panels 1100 and two opposing lower panels 1200. The upper panel 1100 is defined by an upper longitudinal crease 1110 and a side longitudinal crease 1120. The lower panel 1200 is defined by a lower longitudinal crease 1210 and a side longitudinal crease 1120. These panels 1100, 1200 can be made of any suitable, water-resistant, pliable material, such as paper, cardboard, plastic and the like. Alternatively, the interior surfaces or both interior and exterior surfaces of the panels 1100, 1200 can be coated with a thin, water-resistant coating, epoxy or synthetic resin to prevent the urine passing through the device 1000 from leaking through the panels 1100, 1200.

Although many of the prior urination device is made from a biodegradable material that can be flushed downed the toilet after use, such devices are too flimsy and not sturdy enough to provide adequate protection during use. Accordingly, the claimed device 1000 is preferably made of sturdy biodegradable material that can be disposed of in any conventional manner, e.g., a waste receptacle, without any adverse environmental effect.

The device 1000 is preferably constructed from a blank, not shown, wherein longitudinal creases 1110, 1120, 1210 are scored on the blank. The device 1000 is preferably packaged in a closed or a flat configuration as shown in FIG. 1. The flat or closed configuration of the device 1000 enables it to be conveniently carried in a purse or pocketbook, and, therefore, readily available at all times. Preferably, one of the upper panels 1100 comprises a strip (not shown) for attaching the strip to the opposing upper panel 1100 by glue or other comparable means.

Each of the two opposing lower panels 1200 has a first end 1220 and a second end 1230. Preferably, the second end 1230 is concave to provide a tight contoured fit around the woman's vulva when the device is held in place during use. In accordance with an exemplary embodiment of the claimed invention, the second end 1230 comprises a rolling part 1240 to provide a smooth curved second end 1230, thereby eliminating any sharp edges touching the user and alleviating any potential discomfort or irritation to the user's body during use.

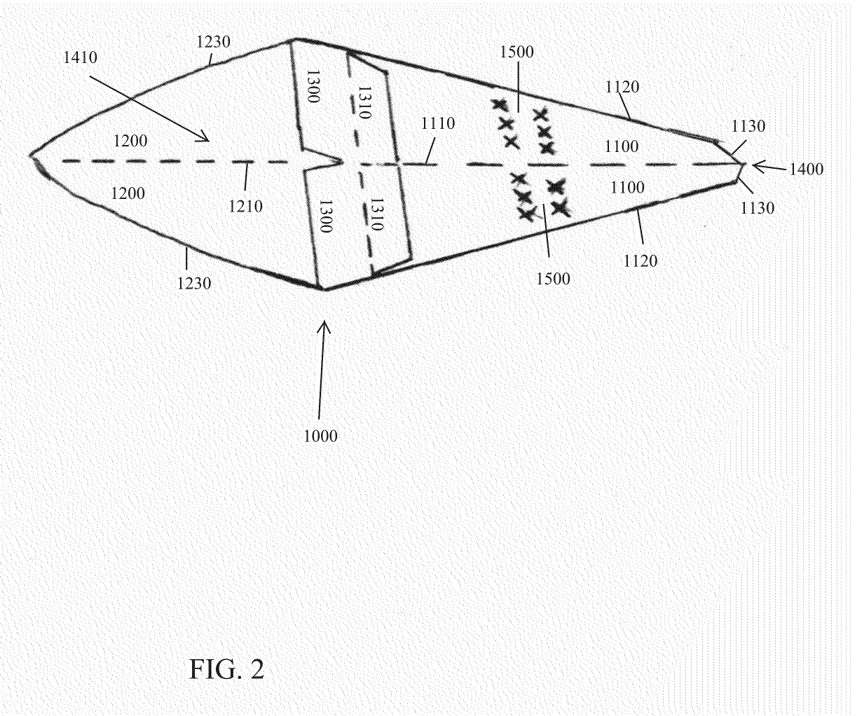

Each of the two opposing upper panels 1100 has a first end 1130 and a second end 1140. In accordance with an exemplary embodiment of the claimed invention, the second end 1140 of the upper panels 1100 comprises a splash guard 1300 to protect the user from accidental leakage or spillage during use. The splash guard 1300 and the rolling part 1240 of the second end 1230 provide a comfortable, sealed perimeter around the vulva to protect the user from accidental leakage or spillage during use. Also, the splash guard 1300 insures that urine is directed towards the interior channel 1420 of the device 1000 and does not splash out of the device 1000. Preferably, the upper panels 1100 additionally comprises a second splash guard 1310 (as shown in FIG. 2) to further protect the user from accidental upward splash, leakage and spillage. Furthermore, the rolling part 1240 and the splash guard 1300 eliminate any sharp edges touching the user, thereby minimizing any potential discomfort or irritation to the user's body during use.

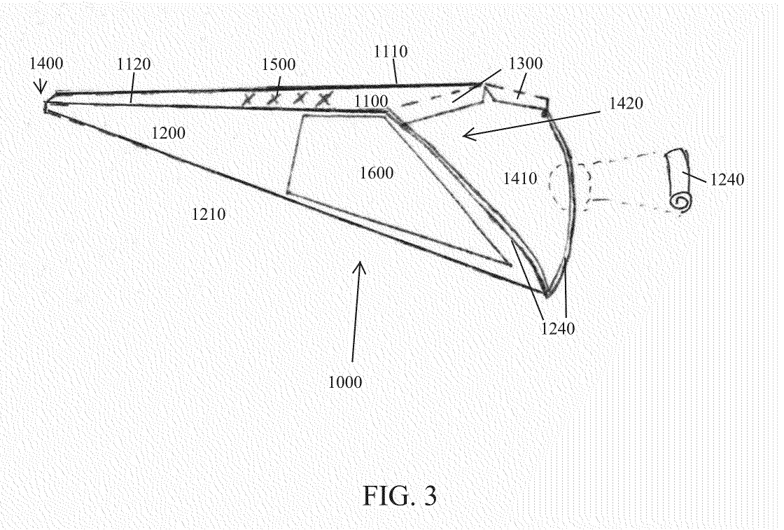

The user applies pressure to both the lower longitudinal crease 1210 and the upper longitudinal crease 1100 of the device 1000 in a closed configuration (see, e.g., FIG. 1) using one hand to open the device 1000 to a useable configuration and deploying the splash guard 1300 (see, e.g., FIGS. 2 and 3). The pressure applied by the fingers of the user's hand pushes the upper longitudinal crease 1100 and the lower longitudinal crease 1210 towards each other and deploys the splash guard 1300 downward or towards the second opening 1410, thereby moving the device from the closed/flat configuration to the opened/useable configuration. It is appreciated that only one hand is needed to open the device 1000, thereby leaving the other hand free. When the device 1000 is opened, the splash guard 1300 deploys to essentially reduces the second opening 1410 slightly to provide an additional wall of protection during use. Additionally, the splash guard 1300 provides a targeted zone to direct the urine towards the interior channel 1420 of the device 1000, thereby protecting the user from accidental leaking or spillage during use.

In the opened/useable configuration, the second ends 1140 of the opposing upper panels 1100, the second ends 1230 of the opposing lower panels 1200 and the splash guard 1300 form a second opening 1410. The second opening 1410 of the device 1000 is placed around the woman's vulva to permit the woman to urinate from a standing position. Preferably, the device 1000 is used such that the splash guard 1300 is at the top of the device 1000 and the opposing lower panels 1200 is at the bottom of the device 1000 to direct the urine to the first opening 1400 and to provide a comfortable, tight contoured fit around the vulva by the rolling part 1240 of the concave second end 1230.

In the opened/useable configuration, the device 1000 resembles a funnel with the first ends 1130 of the opposing upper panels 1100 and the first ends 1220 of the opposing lower panels 1200 forming a first opening 1400. The urine travels down the interior channel 1420 of the device 1000 and exits the device 1000 away from the user through the first opening 1400. Preferably, the first opening is angled such that the opposing upper panels 1100 extends beyond the lower panels 1200 to prevent the exiting urine from splashing upward.

In accordance with an exemplary embodiment of the claimed invention, as shown in FIGS. 1-3, the exterior surfaces of the opposing upper panels 1100 and/or opposing low panels 1200 comprises a gripping portion 1500 to prevent the device 1000 slipping from the user's hand during use. The gripping portion 1500 can comprise a rough surface, a rubberized coating and the like to provide sufficient friction to prevent slippage during use.

In accordance embodiment of the claimed invention, as shown in FIG. 3, the device 100 comprises a sanitary wipe 1600 which is removably attached to the exterior surface of the device 1000, e.g., to the exterior surface of one of the opposing lower panels 1200 or one of the opposing upper panels 1100. The sanitary wipe 1600 is preferably made of biodegradable material. The sanitary wipe 1600 can be peeled off from the device 1000 to dry the area after the user has finished urinating. This provides the user with the additional convenience of carrying an uncontaminated wipe to dry herself with after using the device 1000, particularly in a remote area where no public facilities are available.

Although the claimed invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the claimed invention described in the specification.

1. A disposable device for assisting a female in urinating from a standing position and for directing the urine a suitable distance from the female, comprising:
a first end having an opening for discharging urine;
a second end having an opening for receiving the urine and being larger than the opening in the first end;
a gripping portion on the exterior surface of the device comprising a rough surface or rubberized coating providing friction to prevent slippage and to operate the device with a user's one hand; and
a splash guard extending downward from an upper portion of the second end and covering a portion of the opening in the second end to provide a sealed perimeter at the second end to contain the urine within the device and prevent leakage of the urine.

2. The device of claim 1, wherein the second end further comprises a rolling part to provide a smooth second end to eliminate sharp edges.

3. (canceled)

4. The device of claim 1, further comprising a sanitary wipe removably attached to an exterior surface of the device.

5. The device of claim 1, wherein the splash guard is located at an upper portion of the second end and a rolling part is located at a lower portion of the second end to eliminate sharp edges.

6. The device of claim 5, wherein the lower portion of the second end is concave in shape to provide a tight contoured fit.

7. The device of claim 1, further comprising a second splash guard extending upwardly from the second end to contain the urine within the device and prevent leakage of the urine.

8. The device of claim 1, wherein the device is made from a biodegradable and water-resistant material.

9. The device of claim 1, wherein interior surfaces of the device is coated with water-resistant coating, epoxy or synthetic resin.

10. The device of claim 1, wherein the device is funnel shaped in an open useable configuration.

11. A disposable device for assisting a female in urinating from a standing position and for directing the urine a suitable distance from the female, comprising:
a first end having an opening for discharging urine;
a second end having an opening for receiving the urine and being larger than the opening in the first end;
a gripping portion on the exterior surface of the device comprising a rough surface or rubberized coating providing friction to prevent slippage and to operate the device with a user's one hand; and
a rolling part at the second end to provide a smooth second end to eliminate sharp edges.

12. The device of claim 11, further comprising a splash guard extending downward from an upper portion of the second end and covering a portion of the opening in the second end to provide a sealed perimeter at the second end to contain the urine with the device and prevent leakage of the urine.

13. (canceled)

14. The device of claim 11, further comprising a sanitary wipe removably attached to an exterior surface of the device.

15. The device of claim 12, wherein the splash guard is located at an upper portion of the second end and a rolling part is located at a lower portion of the second end to eliminate sharp edges.

16. The device of claim 14, wherein the lower portion of the second end is concave in shape to provide a tight contoured fit.

17. The device of claim 11, further comprising a second splash guard extending upwardly from the second end to contain the urine within the device and prevent leakage of the urine.

18. The device of claim 11, wherein the device is made from a biodegradable and water-resistant material.

19. The device of claim 11, wherein interior surfaces of the device is coated with water-resistant coating, epoxy or synthetic resin.

20. The device of claim 11, wherein the device is funnel shaped in an open useable configuration.

21. The device of claim 1, further comprising upper and lower longitudinal creases configured to unfold the device to a useable configuration from its a flat or closed configuration in response to a pressure from fingers of a user's one hand pushing the upper and lower longitudinal creases toward each other.

22. The device of claim 11, further comprising upper and lower longitudinal creases configured to unfold the device to a useable configuration from its a flat or closed configuration in response to a pressure from fingers of a user's one hand pushing the upper and lower longitudinal creases toward each other.
